(19) United States
(12) Patent Application Publication
(10) Pub. No.: US 2015/0011895 A1
(43) Pub. Date: Jan. 8, 2015

(54) Methods and Systems for Determining Mechanical Properties of a Tissue

(71) Applicant: University of Washington Through Its Center for Commercialization, Seattle, WA (US)

(72) Inventors: Murray Johnstone, Bainbridge Island, WA (US); Ruikang Wang, Seattle, WA (US)

(21) Appl. No.: 14/371,337

(22) PCT Filed: Mar. 28, 2013
(86) PCT No.: PCT/US2013/034334

§ 371 Date: Jul. 9, 2014

Related U.S. Application Data
(60) Provisional application No. 61/616,962, filed on Mar. 28, 2012.
(60) Provisional application No. 61/780,367, filed on Mar. 13, 2013.

(51) Int. Cl. A61B 5/00

(52) U.S. Cl. CPC A61B 5/0051; A61B 5/0066
USPC 600/476

(57) ABSTRACT

Systems and methods for determining mechanical properties of a biological tissue in a subject are provided. A low coherence optical interferometer detects waves generated from a surface of a tissue in a subject. The waves are generated from elastographic deformation of the tissue induced by an impulse stimulation. Phase velocities can then be determined from the waves, and elastographic properties from the phase velocities, including an elasticity value for a portion of the surface of the tissue.

### CROSS-REFERENCE TO RELATED APPLICATIONS

This application claims priority to U.S. Provisional Patent Application Ser. No. 61/616,962 filed on Mar. 28, 2012, and U.S. Provisional Patent Application Ser. No. 61/780,367 filed on Mar. 13, 2013 which are both hereby incorporated by reference in their entireties.

### BACKGROUND

The alteration of biomechanical properties of tissue is common in many tissue pathologies. Such changes of mechanical properties of biological tissues, especially changes in stiffness, may correlate with a biological tissue's pathological status. Assessing biomechanical properties is thus useful in improving an understanding of tissue pathophysiology, which may aid medical diagnosis and treatment of the pathology.

Skin disorders, diseases, burns, reconstructive tissues, and other conditions may be diagnosed, monitored and subject to prognostic assessment by means of measuring such mechanical properties. Assessment and management decision guidance for appropriate laser, surgical, or pharmacologic intervention for skin conditions such as skin cancers, port wine stain, psoriasis, tissue constructs, and tattoo removals are other examples of conditions where measuring mechanical properties may be beneficial. Still other examples extend to assessment of corneal properties to provide guidance relative to the appropriateness of pharmacologic, laser, or surgical interventions involving the eye. Further examples extend to assessment of tissues of the vascular walls of arteries or veins of a subject in vivo.

Elastography is a biomedical imaging technique that provides elastic properties as well as anatomic information related to biological tissue in a subject. Initial information about the elastic and anatomic properties may be used to then measure changes in the mechanical properties of the same biological tissues.

Imaging techniques have been used to measure a continuous shear wave generated by an external shaker, permitting quantification of the elastic properties of tissue in vivo. The shear wave measurement works if the investigated tissue is mechanically homogeneous, but becomes problematic when the tissue is heterogeneous, e.g. layered structures, which is often the case for in vivo examination of tissues such as human skin. Additionally, prior methods have not been able to assess mechanical properties of a tissue beyond a standard imaging depth of greater than 1.5 mm beneath the tissue surface.

There is a need for a sensitive, non-invasive method and system for assessing the mechanical properties within a heterogeneous biological sample of a subject.

### SUMMARY

In accordance with the present invention, a system and a method are defined for determining mechanical properties of a biological tissue in a subject. In one embodiment, the method may comprise detecting with a low coherence optical interferometer at least one wave generated from a surface of a tissue in a subject, wherein at least one wave is generated from elastographic deformation of the tissue induced by stimulation from an impulse. Phase velocities are then determined from at least one wave, and elastographic properties are determined from the phase velocities, including determining elasticity of a portion of the surface of the tissue. The method may be for diagnosing, providing a prognosis, or monitoring treatment of a tissue disorder, for example.

The detection of at least one wave may comprise detecting a wave traveling in a direction axial or lateral to the surface of the tissue. The impulse stimulation may be a mechanical stimulation, such as a shaker that is applied at an angle with respect to the surface of the tissue, or may be a laser that does not contact the surface of the tissue. Alternatively, a focused acoustic wave force may generate the impulse stimulation.

In one embodiment, a disposable material that is capable of absorbing energy induced by the impulse stimulation is placed on the surface of the tissue.

In another embodiment, an elastographic mapping system is provided. The system comprises an OCT probe, an optical circulator, a stimulator configured to deliver an impulse stimulation to the surface of a tissue, and a physical computer readable storage medium. The physical computer readable storage medium comprises instructions executable to perform functions to acquire a plurality of microstructural images from optical coherence tomography scans of the tissue, detect at least one wave generated from the surface of the tissue, determine measurements of phase velocities from the at least one wave, determine elastographic properties of the surface of the tissue from the measurements, and map the elastographic properties onto the plurality of microstructural images.

The system and method may be used for a subject at risk of any skin pathology, including but not limited to malignant melanoma, scleroderma or other collagen diseases, squamous cell carcinoma or a precursor of squamous cell carcinoma, basal cell carcinoma, and differentiation of actinic keratosis.

The system and method may be used for a subject at risk of any vascular tissue pathology, including but not limited to cardiovascular disease, arteriosclerosis, atherosclerosis, cardiac valve disease, cardiac wall disease, cardiomyopathy, congenital cardiac disorders, aortic aneurism, cerebrovascular disease, renal vascular disease, and peripheral vascular disease.

The system and method may also be used for a subject at risk of any ocular pathology, including but not limited to corneal dystrophies, Fuch's corneal dystrophy, kerataconus, surgery-induced corneal endothelial dysfunction, trauma related corneal injury (both immediately post injury, in the intermediate period, and after stabilized corneal tissue healing and remodeling), basement membrane disease, corneal degenerations, corneal vascularization, corneal scarring, corneal ectasia, anterior, stromal and posterior dystrophies, and corneal edema. The system and method may also be used to assess corneal status prior to, during, and after a surgery selected from the group consisting of: corneal assessment before refractive surgery, corneal assessment after refractive surgery, corneal assessment before cataract surgery, corneal surgery performed to treat a corneal disorder, penetrating keratoplasty, and transplant of any portion of the cornea.

These as well as other aspects and advantages of the synergy achieved by combining the various aspects of this technology, that while not previously disclosed, will become apparent to those of ordinary skill in the art by reading the following detailed description, with reference where appropriate to the accompanying drawings.

### BRIEF DESCRIPTION OF THE FIGURES

FIG. 1 depicts a schematic of an exemplary system in accordance with at least one embodiment:

FIG. 2a depicts a schematic of a sample for use with the exemplary system of FIG. 1 in accordance with at least one embodiment;

FIG. 2b depicts a schematic of a sample for use with the exemplary system of FIG. 1 in accordance with at least one embodiment;

FIG. 2c depicts a schematic of a sample for use with the exemplary system of FIG. 1 in accordance with at least one embodiment;

FIG. 3a depicts a graph illustrating surface waves plotted over time, in accordance with at least one embodiment:

FIG. 3b depicts a graph illustrating surface waves plotted over time, in accordance with at least one embodiment;

FIG. 4a depicts a graph illustrating normalized surface wave amplitude over the location with respect to the stimulator, in accordance with at least one embodiment;

FIG. 4b depicts a graph illustrating normalized surface wave amplitude over the location with respect to the stimulator, in accordance with at least one embodiment;

FIG. 5a depicts a graph illustrating surface waves plotted over time, in accordance with at least one embodiment;

FIG. 5b depicts a graph illustrating surface waves plotted over time, in accordance with at least one embodiment;

FIG. 6 depicts a graph illustrating normalized surface wave amplitude over the location with respect to the stimulator, in accordance with at least one embodiment;

FIG. 7 depicts a graph illustrating phase velocity plotted over frequency, in accordance with at least one embodiment;

FIG. 8 depicts a graph illustrating surface waves plotted over time, in accordance with at least one embodiment;

FIG. 9 depicts a graph illustrating normalized surface wave amplitude over the location with respect to the stimulator, in accordance with at least one embodiment;

FIG. 10 depicts a graph illustrating phase velocity plotted over frequency, in accordance with at least one embodiment; and

FIG. 11 depicts a simplified flow diagram of an example method that may be carried out to measure mechanical properties in a living tissue, in accordance with at least one embodiment.

### DETAILED DESCRIPTION

In the following detailed description, reference is made to the accompanying figures, which form a part thereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

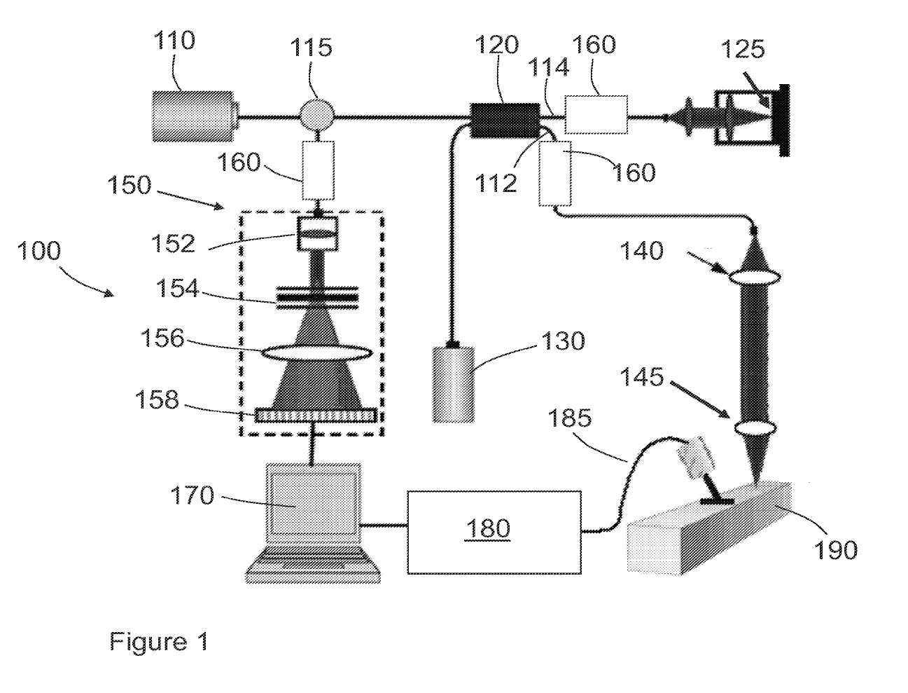

FIG. 1 depicts a schematic of an exemplary system 100 in accordance with at least one embodiment. The system may be used, among other things, to measure biomechanical properties of a living tissue sample of a subject. Thus, the system 100 may be used on a subject in vivo. As referenced herein, a subject may be a human subject.

In FIG. 1, an OCT system is shown as system 100. The system 100 may include a light source 110, a nonreciprocal optical element 115, a fiber coupler 120, a reference mirror 125, a laser diode 130, a collimating lens 140, an objective lens 145, a spectrometer 150 comprising a collimator 152, a diffraction grating 154, a focusing lens 156, and an array detector 158 (e.g., a line scan camera). The system 100 further includes a plurality of polarization controllers 160, a main computing system 170, and a signal generator 180 including a stimulator 185. A sample 190 to be imaged is also shown in FIG. 1.

The OCT system may be a phase-sensitive OCT (PhS-OCT) system, and as discussed in further detail below may be used to independently detect three properties of a sample: (i) waveforms generated by the stimulator 185: (ii) sample scattering properties revealing structure of the sample 190; and (iii) complex motion properties revealing vascular microstructure, simultaneous maps of mechanical properties, morphology, and microcirculation of the sample 190.

In one example embodiment, the light source 110 may be a low temporally coherent light source, such as a broadband superluminescent diode. In other embodiments, other light sources may be used. In one example embodiment, the light source 110 has a central wavelength of about 850-1800 nm The light source may have a central wavelength of about 1310 nm, for example. In one example embodiment, the light source 110 has a spectral bandwidth of about 46 nm.

The nonreciprocal optical element 115 may be an optical circulator, and may have a first port connected to receive light from the light source 110. The nonreciprocal optical element 115 may further include a second port that may direct light from the first port to the fiber coupler 120 and receive light back from the fiber coupler 120, and a third port for directing light received from the fiber coupler 120 to the spectrometer 150.

The fiber coupler 120 serves as a beamsplitter, which transmits or splits some fraction of the power of the incident light power from the light source 110 into each of a sample arm 112 and a reference arm 114. Light returning from both the sample and the reference arms 112 and 114 may be fed to the spectrometer 150 via the nonreciprocal optical element 115. In one example embodiment, the fiber coupler 120 may comprise a pair of fibers partially fused together. The fiber coupler 120 may be a 2×2 fiber coupler.

The reference mirror 125 serves to reflect light directed from the fiber coupler 120 back to the fiber coupler 120.

The laser diode 130 may be a 633 nm laser diode, and serves the purpose of aiming non-visible light at the target during detection of SAWs on the sample 190. The laser diode 130 visually guides the measurement.

The fiber coupler 120 feeds light to a collimating lens 140 of the sample arm 112, which is then focused by the objective lens 145 onto the sample 190. In one example embodiment, the objective lens 145 may comprise a focal length of about 50 mm.

The light source 110 is directed through the nonreciprocal optical element 115 to the fiber coupler 120 which splits the light into the two arms 112 and 114, the reference arm 114 being directed at the reference mirror 125 and the sample arm 112 indicating the OCT probe beam being directed at the sample 190. The OCT probe beam may be either a distance away from or may coincide with the mechanical impulse stimulation. Two or more OCT probes may be placed at a distance from the impulse stimulation.

Light backscattered from the sample 190 in the sample arm 112 is then directed to the fiber coupler 120 and the nonreciprocal optical element 115, along with the reflected light from the reference mirror 125, which is then sent to the spectrometer 150. The spectrometer 150 may then feed the output to the computing system 170 for further processing.

Within the spectrometer 150, the collimator 152 directs the light to the diffraction grating 154, which may serve to split and diffract the light into several light beams that travel in different directions. The focusing lens 156 may serve to focus the light beams received from the diffraction grating 154 into the line CCD 158.

The spectrometer 150 may then send its output to the computing system 170 for further processing. The spectrometer 150 may have an acquisition rate of about 47,000 A-scans per second. The system 100 may be configured for M-mode acquisition, wherein about 2,048 A-scans are acquired to obtain one M-mode scan, from which the phase changes due to the SAWs on the surface of the sample 190.

The signal generator 180 controls the stimulator 185, and may receive a trigger or other signal from the main computing system 170 instructing the signal generator 180 to deliver impulse stimulation via the stimulator 185.

The stimulator 185 may be a mechanical impulse stimulator, and may comprise a shaker or an incident pulsed laser, for example. A shaker may comprise a single element piezoelectric ceramic with a metal rod attached at the end. The metal rod may comprise a length of about 20 mm and a diameter of about 2 mm, in one example, and may serve as a line source. The shaker may be configured to generate a 20 Hz pulse train with a 0.2 percent duty cycle, producing frequency contacts of up to about 10 KHz in the signals at the sample 190 surface.

In another example, the stimulator 185 is a laser that does not contact the sample 190 but instead delivers a laser beam to the sample to excite surface and interior waves from the sample 190. An incident pulsed laser may have a wavelength tuned to be fully or nearly fully absorbed at the surface of the sample 190. The absorbed laser pulse is converted into mechanical energy that generates longitudinal, shear, and surface acoustic waves (SAWs) propagating within or on the surface of the sample 190. In yet another example, the stimulator may be an ultrasound device.

The main computing system 170 may include a processor, data storage, and logic. These elements may be coupled by a system or bus or other mechanism. The processor may include one or more general-purpose processors and/or dedicated processors, and may be configured to perform an analysis on the output from the spectrometer 150. An output interface may be configured to transmit output from the computing system to a display. The computing system 170 may be further configured to send trigger signals to any of the spectrometer 150 and the signal generator 180. Such trigger signals may be sent by the computing system 170 to synchronize the OCT system with the signal generator 180.

The system 100 may provide microstructural images of the sample 190 as a function of depth, allowing for imaging of the sample 190 in addition to detection of SAWs.

In operation, a subject is positioned at a designated location to allow for observation of desired biological tissues of the sample 190. The sample 190 may be a living tissue, and may be observed in vivo. In some example embodiments where contact with the tissue is not a concern, a disposable thin sheet may be placed in surface contact with the sample 190. The thin sheet may be coated with a substance that has high absorption properties at the excitation laser wavelength and facilitates mechanical pulse wave generation within the sample 190.

When the sample 190 is stimulated with an impulse from the stimulator 185, ultrasonic waves are induced, which propagate within the sample 190. Among these waves, P-waves (compression waves) and S-waves (shear waves) travel within the sample 190 while surface waves (Rayleigh waves) travel along the surface of the sample 190. The Rayleigh waves are used to characterize the biomechanical properties (e.g., elastic properties) of the sample 190. Generally, the propagation of a surface wave in a heterogeneous medium (i.e., layered materials) shows dispersive behavior, that is, the different frequency components have different phase velocities. The phase velocity at each frequency is dependent upon the elastic and geometric properties of the sample 190 at different depths.

The relationship between the SAW phase velocity, C_{R}(f), and biomechanical properties can be approximated as:

$$\begin{matrix}\text{?} {C}_{R} ( f ) = \frac{0.87 + 1.12 v}{1 + \text{?}} \sqrt{\frac{E ( f )}{2 ρ ( 1 + \text{?} )}} & Equation 1 \\ \text{?} \text{indicates text missing or illegible when filed} & \end{matrix}$$

where E(f) is the Young modulus at the SAW frequency f, ν the Poisson ratio, and ρ the density of the material. In a soft solid, ν typically varies between 0.3 and 0.5. In one example embodiment, ν may be 0.45 and ρ may be 1060 kg/m³. Compared to a shear wave method, the surface wave method analyzed herein is more sensitive and directly related to the Young modulus.

The SAW frequency f can be converted into depth information for a given sample, as follows:

$$\begin{matrix}\text{?} λ = \frac{{C}_{R}}{f} & Equation 2 \\ \text{?} \text{indicates text missing or illegible when filed} & \end{matrix}$$

where z is the surface wave penetration depth, which is linearly proportional to its wavelength λ.

For a multilayer medium, in which each layer has different elastic properties, the phase velocity of the surface wave is influenced by the mechanical properties of all of the layers into which it penetrates. The minimum depth that can be sensed using the system 100 in combination with the analyses of Equations 1 and 2 is determined by the maximum frequency contained within the detected SAW signal and is defined as follows:

$$\begin{matrix} f \text{?} = \frac{2 \sqrt{2 {C}_{R}}}{\text{?}} & Equation 3 \\ \text{?} \text{indicates text missing or illegible when filed} & \end{matrix}$$

where r_{c} is the radius of the stimulator in an embodiment wherein the stimulator is a mechanical stimulator, such as a shaker described above.

A trigger signal, such as from the main computing device 170 described above, may be given to both the stimulator 185 via the signal generator 180, and the PhS-OCT system in order to fulfill the time-axial synchronization of the SAW signals at each detected location on the sample 190.

After the SAW signals have been acquired by the system 100, calculations may be applied to determine measurements of biomechanical properties of the sample 190. The SAW displacements Δz may be defined as:

$$\begin{matrix} Δ \text{?} = \frac{Δ \text{?}}{4 \text{?}} & Equation 4 \\ \text{?} \text{indicates text missing or illegible when filed} & \end{matrix}$$

where ΔØ is the detected phase change), λ is the central wavelength of the OCT system (1310 nm in the example described above for system 100), and n is the index of refraction of the sample 190. The index of refraction may be about 1.35, in one example. In the example embodiment of FIG. 1, the average amplitude of a generated SAW was typically in the range of about 20-30 nm.

The signal processing procedure to obtain the phase velocity of the SAW signals may be performed as follows. First, the signal's noise is minimized by the use of a Hilbert-Huang method aimed at reducing the high-frequency random noise. Phase velocity dispersion curves can be calculated for SAWs detected at two adjacent positions. The ratio between the phase difference and the ratio of the distance to the wavelength and may be defined as:

$$\begin{matrix} \frac{Δ \text{?}}{2 \text{?}} = ( x 1 - x 2 ) / \text{?} & Equation 5 \\ \text{?} \text{indicates text missing or illegible when filed} & \end{matrix}$$

where ΔØ is the phase difference between two SAW signals at the locations, x1 and x2. This may be calculated from the cross-power spectrum of two SAWs. Thus, the phase velocity (V) of the SAW travelling from x1 and x2 can be expressed as:

V=(x1−x2)×2π×f/ΔØ Equation 6

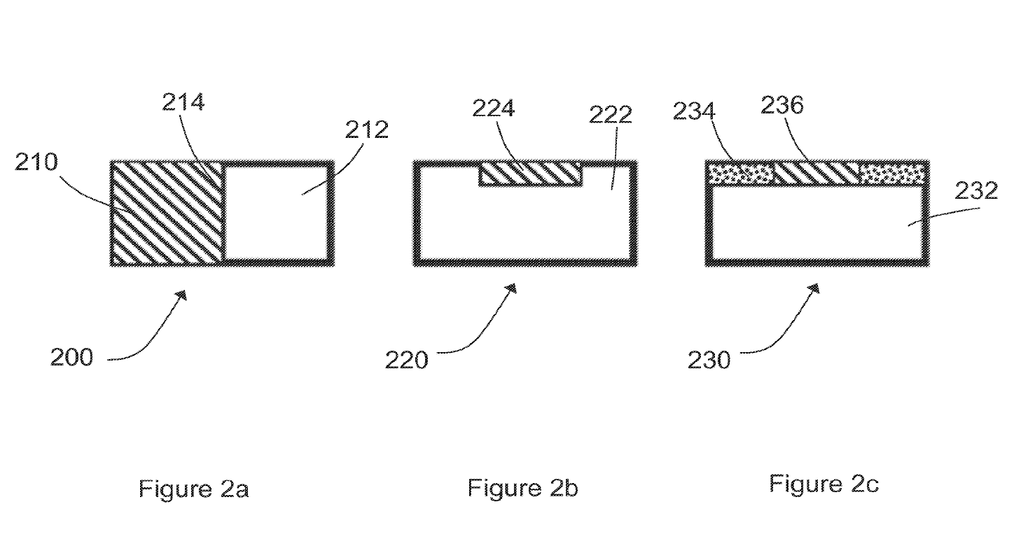

FIGS. 2a-2c are schematics of three example tissue phantoms that may be prepared as samples, such as the sample 190, to simulate the localized change of elasticity (e.g., the alteration of mechanical property in both the lateral and axial directions) when analyzed using a system 100 in combination with Equations 1-6.

FIG. 2a depicts a schematic of a sample 200 for use with the exemplary system 100 of FIG. 1 in accordance with at least one embodiment. The sample 200 may serve as the sample 190 in the system 100, for example. The sample 200 comprises a first material 210 adjacent a second material 212 at an interface 214. The first material 210 has a different elasticity than the second material 212. In the example of FIG. 2a, the first material 210 comprises a homogeneous agar-gel block with a concentration of about 1%, and the second material 212 comprises a homogeneous agar-gel block with a concentration of about 3%.

FIG. 2b depicts a schematic of a sample 220 for use with the exemplary system 100 of FIG. 1 in accordance with at least one embodiment. The sample 220 may serve as the sample 190 in the system 100, for example. The sample 220 comprises a first material 222 and a second material 224 to serve as a localized inclusion within the first material 222. The first material 222 may have a different elasticity than the second material 224.

In one example embodiment, the first material 222 may comprise an agar concentration of about 1%, while the second material 224 comprises an agar concentration of about 3.5%. In another example embodiment, the first material 222 may comprise an agar concentration of about 3.5%, while the second material 224 comprise an agar concentration of about 1%. In yet another example embodiment, the first material 222 may comprise an agar concentration of about 3.5%, while the second material 224 comprise an agar concentration also of about 3.5%.

FIG. 2c depicts a schematic of a sample 230 for use with the exemplary system 100 of FIG. 1 in accordance with at least one embodiment. The sample 230 may serve as the sample 190 in the system 100, for example. The sample 230 comprises a first material 232, a second material 234, and a third material 236. The first material 232 is located under both the second material 234 and the third material 236, and may represent a base layer, comprising in one example embodiment an agar-gel with about 1% agar concentration, and may represent the subcutaneous fat tissue layer of the skin. The second material 234 may comprise an agar-gel with about 1.5-2% agar concentration, and may represent the dermis layer of the skin. The third material 236 may comprise an agar-gel with about 3.5% agar concentration and may represent a lesion or other pathology.

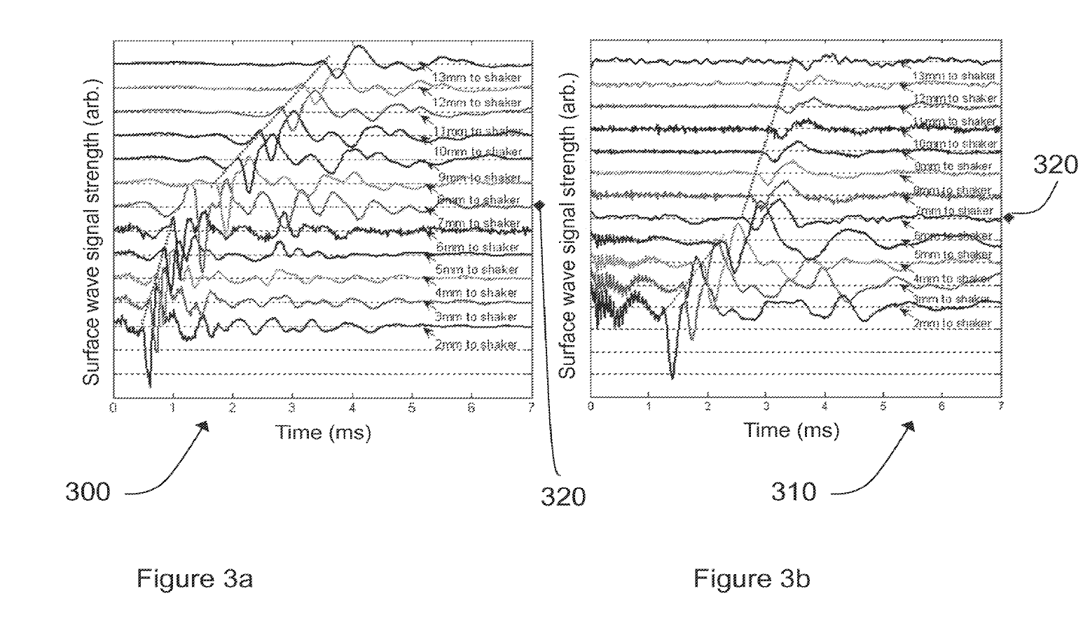

FIG. 3a depicts a graph 300 illustrating surface waves plotted over time, in accordance with at least one embodiment. The surface waves in the graph 300 may be generated from a sample with a configuration such as the sample 200 of FIG. 2a, using a system such as the system 100 of FIG. 1.

Specifically, FIG. 3a plots the surface waves travelling across the interface of the first material 210 to the second material 212 while a stimulator, such as the stimulator 185 of FIG. 1, applies impulse stimulations onto the first material 210.

FIG. 3b depicts a graph 310 illustrating surface waves plotted over time, in accordance with at least one embodiment. FIG. 3b plots the results of surface wave signal strength while a stimulator, such as the stimulator 185 of FIG. 1, applies impulse stimulations onto the second material 212.

For each of the configurations of FIGS. 3a and 3b, the OCT system may detect surface waves first at a position about 2 mm away from the point of stimulation, then sequentially further away from the point of stimulation in 1 mm increments until the detection is 13 mm away from the point of stimulation. A diamond symbol 320 marks the surface wave signal that passes through the interface 214 between the first material 210 and the second material 212.

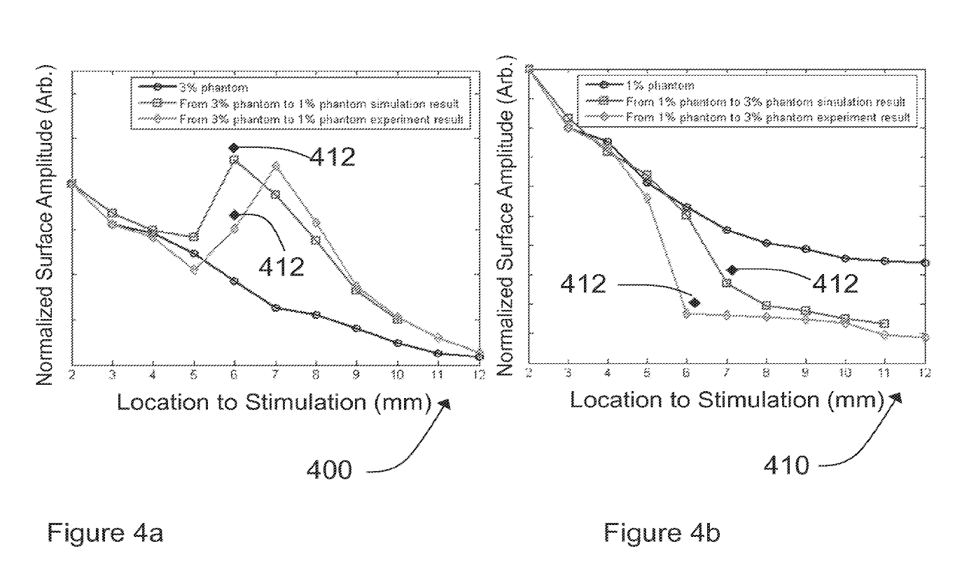

FIGS. 4a-4b plot normalized surface wave amplitude over location with respect to the location of stimulation, and illustrate the SAW amplitudes for the surface waves plotted in FIGS. 3a-3b, respectively. As shown in FIGS. 4a and 4b, for the homogeneous portion of either the first material 210 shown in FIG. 4a or the second material 212 shown in FIG. 4b, the SAW amplitude follows approximately an exponential attenuation when it travels away from its origin (the location of stimulation). After the SAW crosses the interface between the two materials, denoted again with a diamond symbol 412, its amplitude is increased by about 150% when traveling from the first material 210 to the second material 212 (FIG. 4a) and is decreased by about 50% when traveling in the opposite direction, from the second material 212 to the first material 210 (FIG. 4b).

The data in FIGS. 4a-4b illustrates that a system such as the system 100 is sensitive to a lateral change of elasticity in tissues, as measured by either the SAW velocity or the SAW amplitude. When crossing an interface between two materials with differing elasticities, the SAW traveling speed will quickly adapt to that of the material in which it propagates. The abrupt change of the SAW amplitude may be taken as a marker to indicate that the SAW has traveled from tissue with one material property to tissue with another material property and point to the relative location of the material interface relative to the stimulator, serving a potentially useful purpose in biomedical diagnosis both in terms of geometric location of material property changes and composition of the involved tissues.

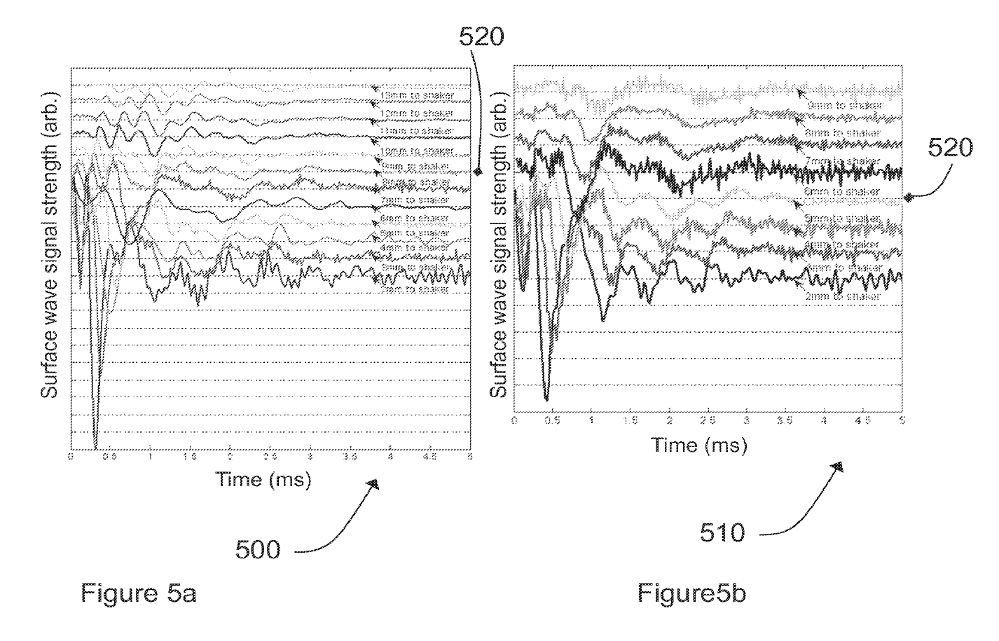

FIG. 5a depicts a graph 500 illustrating surface waves plotted over time for the sample 220 of FIG. 2b, in accordance with at least one embodiment. The sample 220 for the analysis of FIG. 5a has the first material 222 comprising an agar concentration of about 1%, while the second material 224 comprises an agar concentration of about 3.5%.

FIG. 5b depicts a graph 510 illustrating surface waves plotted over time for the sample 220, in accordance with at least one embodiment. The sample 220 for the analysis of FIG. 5b has the first material 222 comprising an agar concentration of about 3.5%, while the second material 224 also comprises an agar concentration of about 3.5%.

For each of the configurations of FIGS. 5a and 5b, the OCT system may detect surface waves first at a position about 2 mm away from the point of stimulation, then sequentially further away from the point of stimulation in 1 mm increments until the detection is 13 mm away from the point of stimulation. A diamond symbol 520 marks the surface wave signal that passes through an interface between the first material 222 and the second material 224.

As shown in FIG. 5a, the surface wave begins to disperse after it crosses the interface between the chicken tissue and the agar, indicating that the SAW was traveling in a heterogeneous medium. In FIG. 5b, in contrast, no dispersion is observed for the transition from first material 222 to the second material 224 (again, where each comprises an agar concentration of about 3.5%), indicating the sample is a homogeneous material.

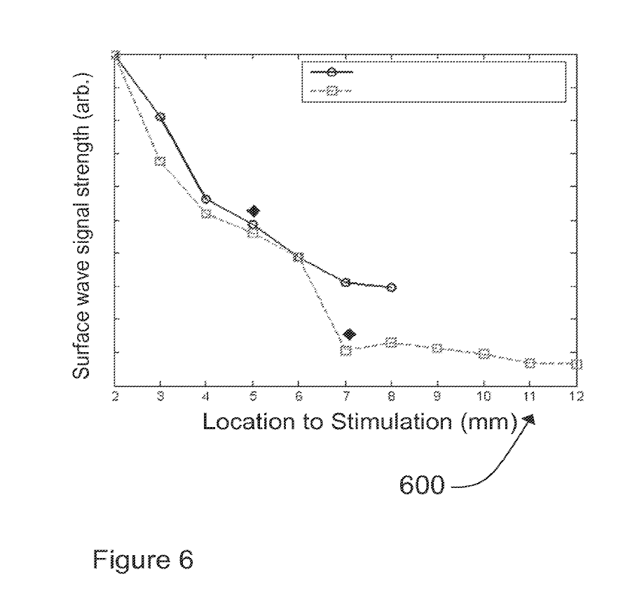

FIG. 6 depicts a graph 600 illustrating normalized surface wave amplitude over the location with respect to the location of the stimulation, in accordance with at least one embodiment. From the data in the graph 600, a significant attenuation is observed when the SAW travels across the boundary between the first material comprising an agar concentration of 1% to the second material comprising an agar concentration of 3.5%. The fact that no abnormal attenuation in SAW amplitude is observed in the graph in FIG. 6 for the sample with two 3.5% agar concentrations indicates that the sample is nearly mechanically homogeneous, and that the boundary in the sample does not have an effect on the proposed system 100 sensitivity to measure the SAW.

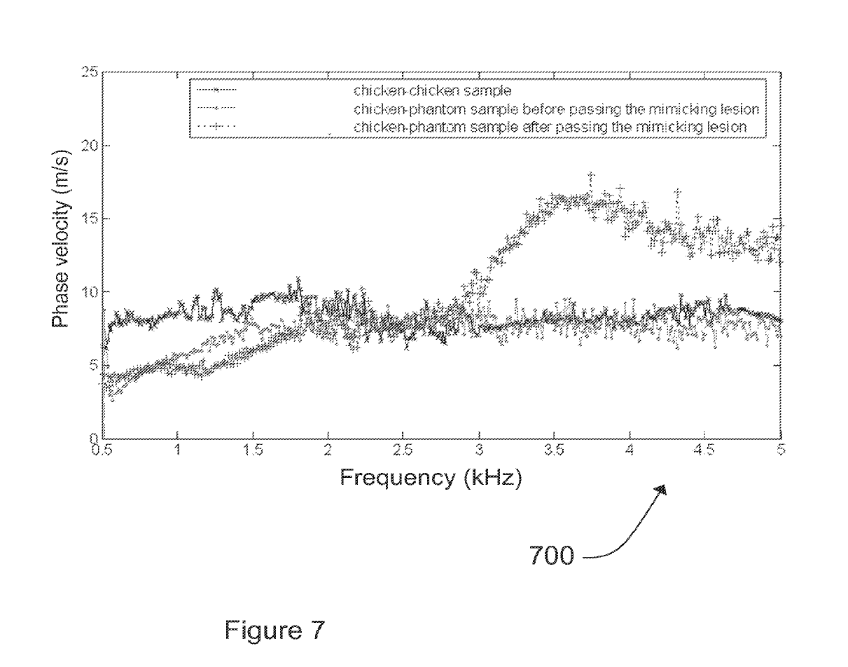

FIG. 7 depicts a graph 700 illustrating phase velocity curves of the SAW for the sample 220 in both configurations analyzed for FIGS. 6a-6b, plotted over frequency, in accordance with at least one embodiment.

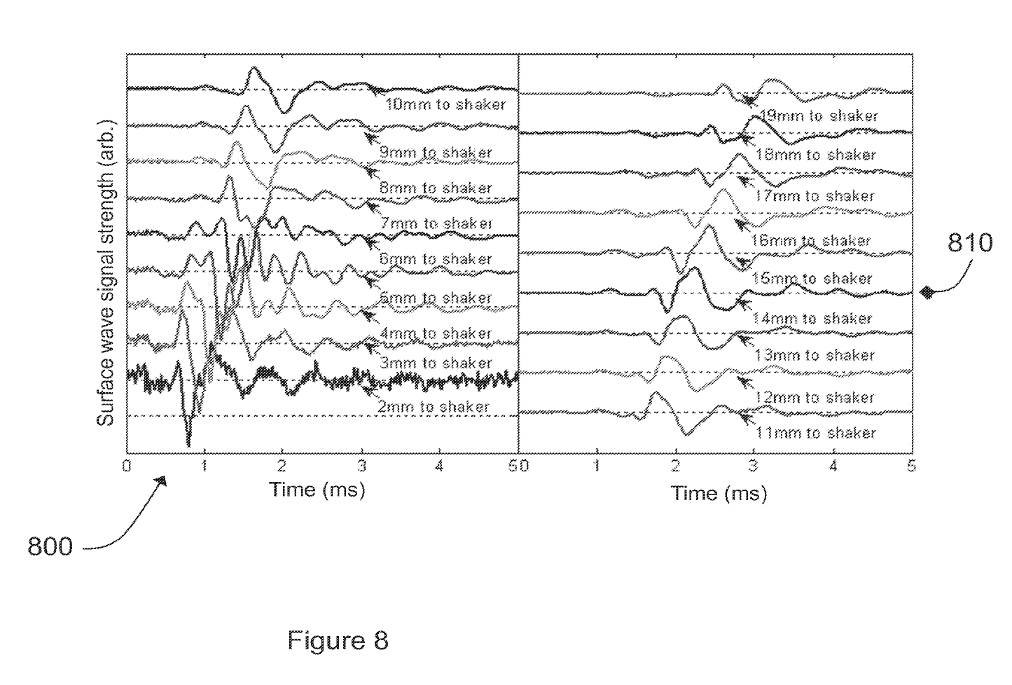

FIG. 8 depicts a graph 800 illustrating surface waves plotted over time for the sample 230 of FIG. 2, in accordance with at least one embodiment. As described with reference to FIG. 2, the first material 232 is located under both the second material 234 and the third material 236, and may represent a base layer, comprising in one example embodiment an agar-gel with about 1% agar concentration. The second material 234 may comprise an agar-gel with about 1.5-2% agar concentration. The third material 236 may comprise an agar-gel with about 3-3.5% agar concentration.

In FIG. 8, the detection points began at a location 2 mm away from the excitation, stepped across the sample 230 at 1 mm increments and finished at a location 19 mm away from the excitation. The surface wave at an interface is denoted by diamond symbol 810.

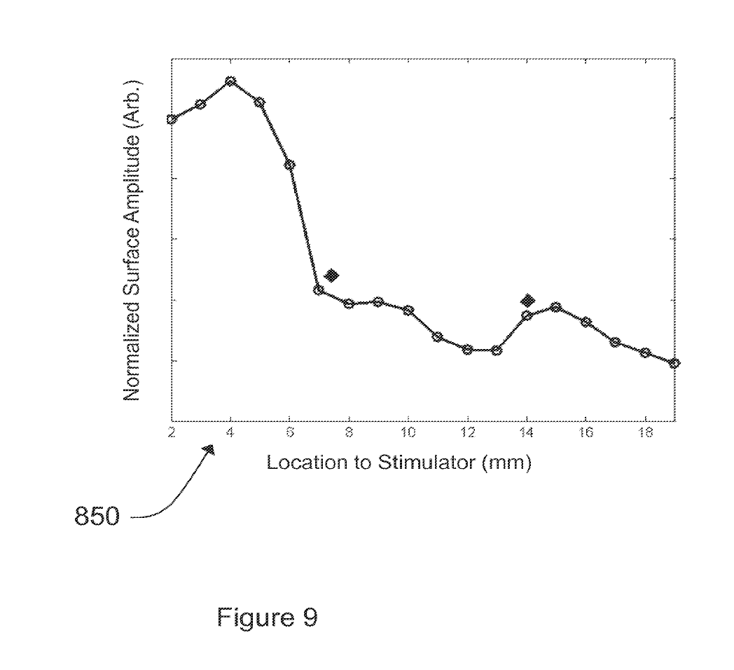

FIG. 9 depicts a graph 850 illustrating normalized surface wave amplitude plotted over the location with respect to the stimulator, in accordance with at least one embodiment, and illustrates the SAW amplitudes for the surface waves plotted in FIG. 8.

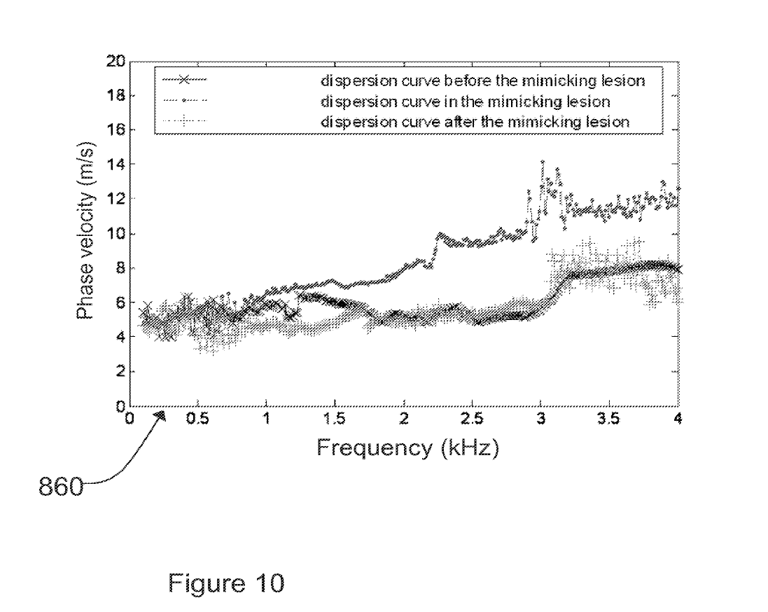

FIG. 10 depicts a graph 860 illustrating phase velocity curves of the SAW for the sample 230 plotted over frequency (kHz), in accordance with at least one embodiment.

The results of the experiments carried out for FIGS. 3a-10, implementing a system such as the system 100 of FIG. 1, demonstrate that with PhS-OCT as a pressure sensor, the SAW is highly sensitive to the elasticity change of a sample in both the vertical and the lateral directions, providing for useful clinical applications in situations where localized quantitative elasticity tissues can be used to detect, aid in diagnosis and provide guidance for treatment of disease processes.

The system and method may be used for a subject at risk of any skin pathology, including but not limited to malignant melanoma, scleroderma or other collagen diseases, squamous cell carcinoma or a precursor of squamous cell carcinoma, basal cell carcinoma and differentiation of actinic keratosis.

The system and method may be used for a subject at risk of any vascular tissue pathology, including but not limited to cardiovascular disease, arteriosclerosis, atherosclerosis, cardiac valve disease, cardiac wall disease, cardiomyopathy, congenital cardiac disorders, aortic aneurism, cerebrovascular disease, renal vascular disease, and peripheral vascular disease.

In an embodiment directed to determining properties of a vascular tissue, an OCT probe containing a detector may enter the subject's body cavities either through an orifice or percutaneously. The impulse stimulus may take two forms: i) an intrinsic physiologic mechanism wherein the cardiac pulse induces a stimulus; or ii) the probe containing the detector also contains an excitation source, such as a mechanical, ultrasound or laser source for example, that delivers the appropriate energy to initiate a stimulus, or provides a stimulus by the subject's natural blood pulse due to heart beat, or by other excitation energy sources. The source of the excitation energy and the detection system may each be contained in a single, in two, or in multiple probes.

The system and method may also be used for a subject at risk of any ocular pathology, including but not limited to corneal dystrophies, fuchs corneal dystrophy, kerataconus, surgery-induced corneal endothelial dysfunction, trauma related corneal injury (both immediately post injury, in the intermediate period, and after stabilized corneal tissue healing and remodeling), basement membrane disease, corneal degenerations, corneal vascularization, corneal scarring, corneal ectasia, anterior, stromal and posterior dystrophies, and corneal edema. The system and method may also be used to assess the corneal status prior to, during, and after a surgery selected from the group consisting of: corneal assessment before refractive surgery, corneal assessment after refractive surgery, corneal assessment before cataract surgery, corneal surgery performed to treat a corneal disorder, penetrating keratoplasty, and transplant of any portion of the cornea.

Glaucoma is caused by elevated intraocular pressure, and currently uses pressure measurement tools that rely on applying an applanation or flattening force to the corneal surface to determine intraocular pressure. Such measurements are highly dependent on assumptions related to mechanical properties of the cornea, which are currently not well characterized for purposes of making adjustments to accurately reflect true intraocular pressure, limiting optimal management of glaucoma.

Because corneal mechanical properties vary considerably between individuals, recorded measurements often do not reflect true intraocular pressure. Glaucoma treatment decisions thus are guided by faulty and often misleading information. Every mm of pressure is believed to impact the course of the disease, thus, the inability to accurately assess pressure puts every patient at risk for progressive permanent vision loss because treatment decisions are based on this imprecise data.

This limited understanding of important mechanical properties of the cornea can hinder diagnosis and successful treatment of any problems in the tissue. An ability to accurately measure tissue changes is also important for quantitative assessment of the tissue properties, changes in properties as a result of disease processes, and subsequent diagnosis, prognosis, or treatment of any issues or functional abnormalities associated with the tissue.

When measurements such as those described with reference to FIGS. 3a-10 are made to determine mechanical properties of a tissue such as the cornea, they can provide a correction factor for independent intraocular pressure measurements that require such correction factors based on corneal mechanical properties. Such correction factors may take the form of a nomogram based on mechanical property measurements, for example. The correction factor is provided by the measured elasticity of the cornea.

The measurement of tissue motion may be used to diagnose, provide a prognosis, monitor treatment and guide treatment decisions for a disorder of the sample of a subject. The treatment may include medical, laser, or surgical intervention.

A treatment decision may be based on the prognosis, monitoring or assessment of current properties of the tissues or regions of the tissue conducted in accordance with the measurement calculated with reference to FIG. 1.

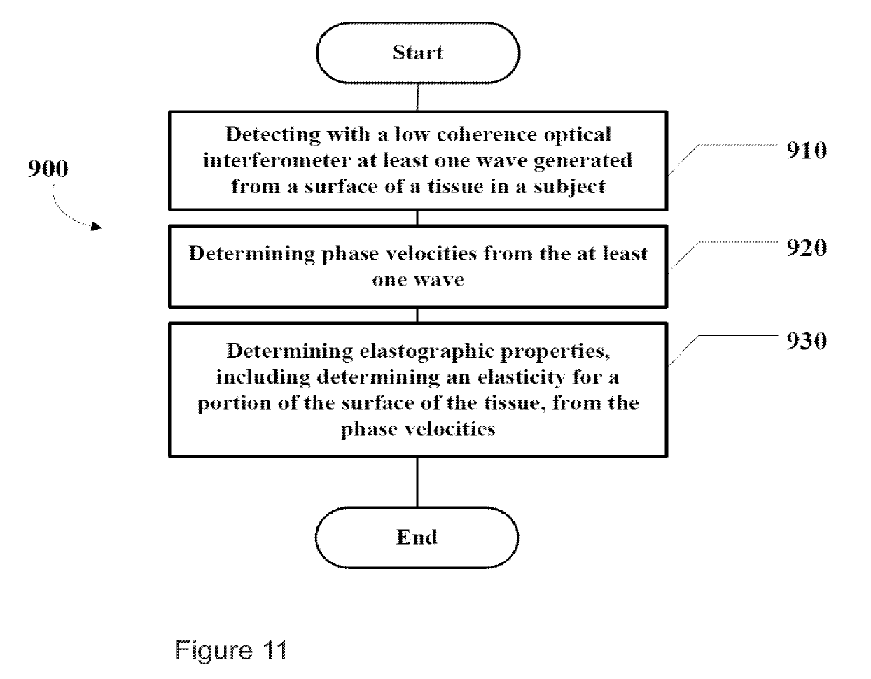

FIG. 11 depicts a simplified flow diagram of an example method 900 that may be carried out to measure elastographic properties in a tissue, in accordance with at least one embodiment. Method 900 shown in FIG. 11 presents an embodiment of a method that, for example, could be used with the system 100.

In addition, for the method 900 and other processes and methods disclosed herein, the flowchart shows functionality and operation of one possible implementation of the present embodiments. In this regard, each block may represent a module, a segment, or a portion of program code, which includes one or more instructions executable by a processor for implementing specific logical functions or steps in the process. The program code may be stored on any type of computer readable medium, for example, such as a storage device including a disk or hard drive. The computer readable medium may include a physical and/or non-transitory computer readable medium, for example, such as computer-readable media that stores data for short periods of time like register memory, processor cache and Random Access Memory (RAW. The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a computer readable storage medium, a tangible storage device, or other article of manufacture, for example. Alternatively, program code, instructions, and/or data structures may be transmitted via a communications network via a propagated signal on a propagation medium (e.g., electromagnetic wave(s), sound wave(s), etc.).

The method 900 allows for determining elasticity of a tissue in a subject. The method 900 may be used to diagnose, develop a prognosis, or monitor treatment for a disorder of the living tissue.

Initially, the method 900 includes detecting with a low coherence optical interferometer at least one wave generated from a surface of a tissue in a subject, at block 910. At least one wave is generated from elastographic deformation of the tissue induced by an impulse stimulation. The impulse stimulation may be delivered from a stimulator such as the stimulator 185 of FIG. 1, in one example embodiment.

The method 900 then includes determining phase velocities from at least one wave, at block 920. The phase velocities may be determined as described with reference to the Equations 1-6 and the examples described with reference to FIGS. 2a-10.

The method 900 includes determining elastographic properties, including determining an elasticity for a portion of the surface of the tissue, from the phase velocities, at block 930.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims, along with the full scope of equivalents to which such claims are entitled. It is also to be understood that the terminology used herein is for the purpose of describing particular embodiments only, and is not intended to be limiting.

1. A method for determining elasticity of a tissue in a subject comprising:
detecting with a low coherence optical interferometer at least one wave generated from a surface of a tissue in a subject, wherein the at least one wave is generated from elastographic deformation of the tissue induced by an impulse stimulation;
determining phase velocities from the at least one wave; and
determining elastographic properties, including determining an elasticity for a portion of the surface of the tissue, from the phase velocities.

2. (canceled)

3. The method of claim 1, wherein detecting at least one wave comprises detecting a wave traveling in a direction axial or lateral to the surface of the tissue.

4. (canceled)

5. The method of claim 1, wherein the impulse stimulation is a mechanical stimulation.

6. The method of claim 5, wherein a shaker comprising a signal generator and a single element piezoelectric ceramic with a line source generates the impulse stimulation.

7. The method of claim 6, wherein the shaker is applied at an angle with respect to the surface of the tissue.

8. The method of claim 1, wherein a laser generates the impulse stimulation.

9. The method of claim 8, wherein the laser does not contact the surface of the tissue.

10. The method of claim 1, wherein a focused acoustic wave force generates the impulse stimulation.

11. The method of claim 1, wherein a disposable material capable of absorbing excitation energy induced by the impulse stimulation is on the surface of the tissue.

12. The method of claim 1, wherein the method is used to diagnose, provide a prognosis, or monitor treatment for a disorder of the tissue.

13. The method of claim 12, wherein the subject is at risk of a skin pathology or has a skin pathology.

14. The method of claim 13, wherein the skin pathology is selected from the group consisting of malignant melanoma, scleroderma or other collagen diseases, squamous cell carcinoma, a precursor to squamous cell carcinoma, basal cell carcinoma, and differentiation of actinic keratosis.

15. The method of claim 12, wherein the subject is at risk of a vascular tissue pathology.

16. The method of claim 15, wherein the vascular tissue pathology is selected from the group consisting of: cardiovascular disease, arteriosclerosis, atherosclerosis, cardiac valve disease, cardiac wall disease, cardiomyopathy, congenital cardiac disorders, aortic aneurism, cerebrovascular disease, renal vascular disease, and peripheral vascular disease.

17. The method of claim 1, wherein the tissue is an ocular tissue, further comprising:
providing a correction factor for independent intraocular pressure measurements based on corneal mechanical properties.

18. The method of claim 1, wherein the tissue is a corneal tissue and the method is used to assess corneal pathologies selected from the group consisting of: corneal dystrophies, fuchs corneal dystrophy, kerataconus, surgery-induced corneal endothelial dysfunction, trauma related corneal injury, basement membrane disease, corneal degenerations, corneal vascularization, corneal scarring, corneal ectasia, anterior, stromal and posterior dystrophies, and corneal edema.

19. The method of claim 1, wherein the tissue is a corneal tissue and the method is used to assess corneal status prior to, during, and after a surgery selected from the group consisting of: corneal assessment before refractive surgery, corneal assessment after refractive surgery, corneal assessment before cataract surgery, corneal surgery performed to treat a corneal disorder, penetrating keratoplasty, and transplant of any portion of the corneal.

20. The method of claim 15, wherein the impulse stimulation is detected by a probe that enters the bloodstream by a percutaneous entry into a blood vessel, and wherein the impulse stimulation is obtained by a blood pulse wave from a heart beat of the subject.

21. The method of claim 1, further comprising:
generating surface wave phase velocity curves from the phase velocities; and
providing elasticity values for portions of the tissue from the surface wave phase velocity curves.

22. The method of claim 1, further comprising:
acquiring a plurality of microstructural images from optical coherence tomography scans of the tissue.

23. The method of claim 23, further comprising:
mapping the elastographic properties of the tissue onto the acquired microstructural images of the tissue.

24. An elastographic mapping system, comprising:
an optical coherence tomography probe;
a stimulator configured to deliver an impulse stimulation to a surface of a tissue; and
a physical computer-readable storage medium;
wherein the physical computer-readable storage medium has stored thereon instructions executable by a device to cause the device to perform functions comprising:
acquiring a plurality of microstructural images from optical coherence tomography scans of the tissue;
detecting at least one wave generated from the surface of the tissue;
determining measurements of phase velocities from the at least one wave;
determining elastographic properties of the surface of the tissue from the measurements; and
mapping the elastographic properties onto the plurality of microstructural images.

25. The system of claim 25, wherein the stimulator is a shaker, a laser, or an ultrasound device.

26.-27. (canceled)

28. An article of manufacture including a tangible computer-readable media having computer-readable instructions encoded thereon, the instructions comprising:
detecting with a low coherence optical interferometer at least one wave generated from a surface of a tissue in a subject, wherein the at least one wave is generated from elastographic deformation of the tissue induced by an impulse stimulation;
determining phase velocities from the at least one wave; and
determining elastographic properties, including determining an elasticity for a portion of the surface of the tissue, from the phase velocities.

29. The article of manufacture of claim 28, wherein the instructions are further executable to perform functions comprising:
acquiring a plurality of microstructural images from optical coherence tomography scans of the tissue; and
mapping the elastographic properties onto the plurality of microstructural images.
